(19) United States
(12) Patent Application Publication
(10) Pub. No.: US 2003/0233018 A1
(43) Pub. Date: Dec. 18, 2003

(54) Method for isomerizing a mixed olefin feedstock to 1-olefin

(76) Inventors: Stephen H. Brown, Brussells (BE); Stephen N. Vaughn, Kingwood, TX (US); Jose Guadalupe Santiesteban, Bethlehem, PA (US); Karl G. Strohmaier, Port Murray, NJ (US)

Correspondence Address: EXXONMOBIL CHEMICAL COMPANY, P O BOX 2149, BAYTOWN, TX, 77522-2149, US

(21) Appl. No.: 10/174,022

(22) Filed: Jun. 18, 2002

(51) Int. Cl. C07C 5/25; C07C 5/23

(52) U.S. Cl. 585/664

(57) ABSTRACT

A method of making 1-olefin such as 1-butene by contacting a mixed olefin feedstock preferably with a small pore molecular sieve catalyst, especially SAPO-34, at a temperature from about 300° C. to about 700° C., and an effective pressure and WHSV to form an olefin product with a 1-olefin:isoolefin conversion index greater than 1:1. A mixed olefin feedstock produced from an oxygenate to olefin process is particularly well suited for the production of 1-olefin.

### FIELD OF THE INVENTION

This invention relates to a system of isomerizing mixed olefins to 1-olefins preferably isomerizing mixed butenes to 1-butenes using a small pore molecular sieve catalyst.

### BACKGROUND OF THE INVENTION

Olefin feedstocks are used to produce a variety of commercially important products including fuels, polymers, plasticizers, and other chemical products. For example, a butene feedstock that contains an isomeric mixture of 1-butene, cis and trans 2-butenes, and isobutene is used to make alkylate fuels, a gasoline additive known as methyl-t-butyl ether (MTBE), and linear low-density polyethylene. The 2-butenes are the most desirable isomers for the production of alkylate. Isobutene is used primarily to make MTBE, and 1-butene can be used as a co-monomer for making linear low-density polyethylene or as a monomer in polybutene production. The worldwide market for 1-butene is approaching 1 billion pounds per year. As a result, the need for each isomeric butene is determined by the desired commercial product.

Catalytic olefin isomerization can be used to alter the ratio of olefin isomers in an olefin feedstock. Olefin isomerization processes use catalysts containing an ammonium phosphate, see, e.g., U.S. Pat. No. 2,537,283, or a precipitated aluminum phosphate within a silica gel, see, e.g., U.S. Pat. No. 3,211,801, to convert 1-butene to 2-butene. U.S. Pat. Nos. 3,270,085 and 3,327,014 are directed to an olefin isomerization process using a chromium-nickel phosphate catalyst. Zeolitic catalysts can also be used to isomerize an olefin stream. However, in a majority of these cases the 1-olefin is converted to the 2-olefin. European Patent Application 0 247 802 discloses using zeolites which include ZSM-22 and ZSM-23 at temperatures of 200° C. to 550° C. to convert 1-butene to 2-butene. The product selectivity of the converted 1-butene is about 92% 2-butene and about 8% isobutene. U.S. Pat. No. 4,749,819 discloses that ZSM-12 and ZSM-48 can also be used to isomerize 1-butene to 2-butenes.

Medium pore non-zeolitic molecular sieve catalysts have also been reported to isomerize mixed olefin feedstock. U.S. Pat. No. 5,132,484 to Gajda, which is incorporated herein by reference, discloses the use of SAPO-11 to convert 2-butene to isobutene or 1-butene. If isobutene is the desired product an operating temperature of from 200° C. to 600° C., preferably from 250° C. to 400° C., is used. If 1-butene is the desired product an operating temperature of from 50° C. to 300° C. is used.

U.S. Pat. No. 5,990,369 to Barger et al., which is incorporated herein by reference, also discloses using SAPO-11 as one of the preferred non-zeolitic molecular sieve catalysts to isomerize 2-butene to 1-butene in a distillation unit containing an isomerization zone. The operating conditions for the isomerization include a temperature ranging from 50° C. to 300° C., a pressure ranging from 100 kPa to 7 Mpa, a LHSV (liquid hourly space velocity) ranging from 0.2 to 10 hr⁻¹, and a hydrogen to hydrocarbon molar ratio of from 0.5 to 10.

U.S. Pat. No. 6,005,150 to Vora, which is incorporated herein by reference, discloses using SAPO-11 in a catalytic distillation unit containing a lower isomerization zone and an upper etherification zone. In the lower zone, 2-butene is isomerized to 1-butene which moves up the column and exits the column in a side draw stream. In the upper zone, the isobutene is catalytically converted to MTBE which is removed in the bottoms stream.

Olefm isomerization is still hampered by the relatively low product selectivity of the isomerized product. As a result, a need exists for a catalytic isomerization process that exhibits a relatively high product selectivity to the desired 1-olefin. Also, a need exists for a process that exhibits a relatively low product selectivity to isoolefin.

### SUMMARY OF THE INVENTION

The present invention provides an isomerization process resulting in a relatively high selectivity of 1-olefin over a small pore molecular sieve catalyst. The method includes contacting an olefin feedstock with a small pore molecular sieve catalyst under conditions effective to isomerize at least a portion of the olefin feedstock to 1-olefin. In one embodiment, the olefin feedstock comprises 1-butene, 2-butene, isobutene and/or butadiene.

In one embodiment, the 1-olefin product is selected from 1-butene, 1-pentene, 1-hexene, 1-heptene, 1-octene, and mixtures thereof. The conditions are selected to provide a 1-olefin:isoolefin conversion index greater than 1:1, 5:1, 10:1, 20:1, or 50:1. The conditions include a temperature of at least or greater than 300° C., 350° C., 400° C., 450° C., or 500° C. The temperature is in a range of from 300° C. to 700° C., 400° C. to 650° C., 450° C. to 600° C., or 450° C. to 550° C. The pressure is from about 5 psia to about 150 psia, and the weight hourly space velocity (WHSV) is from 1 hr⁻¹ to 200 hr⁻¹. With the inventive method, less than 2%, more preferably less than 1%, by weight of the feedstock is converted to hydrocarbons with a higher carbon number.

The small pore molecular sieve catalyst of one embodiment is selected from SAPO-34, CHA, Erionite, Offretite and ZSM-34. The olefin feedstock includes olefin produced by a gas cracking unit, an oxygenate to olefin unit, or a mixture thereof. Preferably, the feedstock is isoolefin depleted and includes less than 1% by weight isoolefin. The olefin feedstock preferably contains a mixture of hydrocarbons with an average carbon number of 4 to 8. Preferably, less than 2% by weight of the feedstock is converted to aromatic hydrocarbons.

In one embodiment of the present invention, the olefin feedstock includes butadiene. This method, optionally includes contacting the butadiene with hydrogen under conditions effective to convert at least a portion of the butadiene to linear butenes. The olefin feedstock can sometimes include isoolefin. If the feedstock includes isoolefin, the inventive method typically includes contacting the isoolefin with an alcohol under conditions effective to convert at least a portion of the isoolefin to an alkyl ether. The alkyl ether is then separated from the olefin feedstock by conventional techniques. Isobutene dimerization or hydration, are alternative methods to separate the isobutene from the olefin feedstock.

Another embodiment of the present invention is a method for isomerizing an olefin feedstock at high temperatures to form 1-olefin. The method includes contacting the olefin feedstock with a molecular sieve catalyst under conditions effective to isomerize at least a portion of the feedstock to 1-olefin. The method of this embodiment has a feedstock which optionally includes 1-butene, 2-butene, isobutene and/or butadiene. The contacting preferably occurs at a temperature of at least or greater than 300° C., 350° C., 400° C., 450° C., or 500° C. Preferably, the conditions are effective to provide a 1-olefin:isoolefin conversion index greater than 1:1, 5:1, 10:1, 20:1, or 50:1. The catalyst of one embodiment is a small, medium or large pore molecular sieve catalyst. Also, the catalyst is a zeolitic catalyst in one embodiment. The catalyst of another embodiment can be a non-zeolitic catalyst and in a more particular embodiment is selected from SAPO-11, SAPO-34, CHA, Erionite, Offretite, ZSM-5 and ZSM-34. Ideally the temperature is in a range of from 300° C. to 700° C., 400° C. to 650° C., 450° C. to 600° C., or 450° C. to 550° C.

The foregoing invention and all its embodiments is more particularily understood by reference to the detailed description of the invention when taken together with the drawings and examples.

### BRIEF DESCRIPTION OF THE DRAWINGS

FIG. 1 is a process scheme of an embodiment of the present invention; and

FIG. 2 is a process scheme of another embodiment of the present invention.

### DETAILED DESCRIPTION OF THE INVENTION

The present invention provides for the isomerization of an olefin feedstock, which contains one or more of 1-olefin, internal olefins, and/or isoolefin, to a 1-olefin by using preferably a small pore molecular sieve catalyst. As used herein, the term “isomerize” includes the metathesis of one linear olefin, e.g., 2-butene, to another linear olefin, e.g., 1-butene. The 1-olefin produced by the process preferably is 1-butene. In other embodiments, the 1-olefin produced is selected from 1-pentene, 1-hexene, 1-heptene, 1-octene, 1-nonene, 1-decene, and mixtures thereof. However, isomerizing a mixed C₅⁺ olefin feedstock to C₅⁺ 1-olefin is exponentially more difficult as the number of carbons increases. This is due, in part, to the increasing number of possible isomers as the number of carbons increases.

Another embodiment of the present invention is a method of isomerizing an olefin feedstock including contacting the olefin feedstock with a molecular sieve catalyst under conditions effective to isomerize at least a portion of the olefin feedstock to 1-olefin. In this embodiment, the contacting preferably occurs at a temperature of at least 300° C. and provides a 1-olefin:isoolefin conversion index of greater than 1:1. The catalyst can include small, medium or large pore molecular sieves.

In accordance with the present invention, the olefin feedstock contacts preferably a small pore molecular sieve catalyst under conditions effective to isomerize at least a portion of the olefin feedstock to 1-olefin. Small pore silicoaluminophosphate (SAPO) molecular sieve catalysts such as SAPO-34 are particularly preferred in the present invention. Desirably, the olefin product will contain a relatively high product ratio of 1-olefin to isoolefin. Then, the 1-olefin produced according to the invention can be used to make a variety of commercial products including linear, low density polyethylene and polybutylene.

Preferrably, the olefin feedstock, of one embodiment, contains one or more types of olefin of the same or different carbon number. For example, an olefin feedstock that contains primarily butenes also includes a mixture of 1-butene and cis- and trans-2-butene. This butene feedstock also contains one or more of the following: isobutene, butanes, isobutane, propylene, propane, pentenes, and other hydrocarbons including oxygenated hydrocarbons. Alternatively, an olefin feedstock contains primarily cis- and trans-2-butenes, for example, if the 1-butene was separated prior to directing the feedstock to the isomerization unit.

The process can be carried out using a wide variety of mixed olefin feedstocks. However, it is preferred that an isoolefin-depleted mixed olefin feedstock containing primarily normal olefins be used in the process. As used herein, the term “isoolefin-depleted” means a mixed olefin feedstock that contains less than about 5% by weight, preferably less than about 2% by weight, more preferably less than 1% by weight, isoolefin. These isoolefin-depleted feedstocks, in some instances, are produced by an olefin process optimized to the production of normal butenes. However, more typically, the isoolefin-depleted feedstock will be provided by first directing an olefin stream to an isoolefin removal unit.

As used herein, the term “isoolefin removal unit” is intended to broadly encompass separation or conversion zones, which result in the removal of isoolefin from an olefin stream fed to that zone with a high degree of selectivity. Examples of such isoolefin removal units include but are not limited to a cold acid extraction process, adsorptive separation, and reaction zones including hydration zones used to produce alcohol or etherification zones. An etherification zone or unit is implemented in one embodiment of the present invention to remove isoolefin from an olefin stream producing a branched alkyl ether product and a mixed olefin feedstock with only small amounts of isoolefin, e.g., less than 3% by weight, preferably less than 2% or less than 1% by weight, isoolefin. The alkyl ether product is removed from the feedstock through known separation techniques. U.S. Pat. No. 4,605,787 to Chu et al., the entirety of which is incorporated herein by reference, provides an example of etherification of isobutene with methanol to produce MTBE in high conversion and selectivity.

Additionally or alternatively, it is preferred that the mixed olefin feedstock be diene-depleted. As used herein, the term “diene-depleted” characterizes a mixed olefin feedstock that contains less than about 5% by weight, preferably less than about 2% by weight, more preferably less than 1% by weight, diene. These diene-depleted feedstocks can be produced by an olefin process optimized to the production of normal butenes. However, more typically, the isoolefin-depleted feedstock is provided by first directing an olefin stream to an diene removal unit.

The term “diene removal unit” is defined herein is intended to broadly encompass separation or conversion zones which with a high degree of selectivity result in the removal (or conversion) of dienes such as butadiene from an olefin stream fed to that zone. An example of a diene removal unit is a diene hydrofiner wherein the diene is hydrogenated across one double bond to convert diolefin to monoolefin, such as 1-butene. Dienes are optionally removed by selective hydrogenation in the presence of a solid catalyst comprising nickel and a noble metal such as platinum or palladium or silver as disclosed in U.S. Pat. No. 4,409,410, the entirety of which is incorporated herein by reference. In this embodiment, the diene contacts a selective hydrogenation catalyst in a reaction zone to produce additional 1-olefin and/or internal olefin. The additional internal butenes are further converted to 1-butene through catalytic isomerization. Alternatively, the diene is removed by known oligimerization or polymerization techniques.

In one embodiment, an olefin feedstock containing primarily butenes is used. This olefin feedstock optionally contains saturated hydrocarbons, C₁ to C₃ hydrocarbons, and C₅⁺ hydrocarbons. It is preferred that this mixed olefin feedstock will contain at least 15% by weight, preferably at least 25% by weight, more preferably 35% by weight and most preferably greater than 50% by weight 2-butenes.

In another embodiment, a C₅ cut containing 1-pentene, 2-pentene, isopentene, pentane and isopentane is used. A suitable feedstock includes a C₅ cut from a gas cracking unit, particularly a C₅ cut in which the isopentene has been removed by an etherification unit. In the etherification unit the isopentenes in the C₅ cut reacts with an alcohol to form an alkyl tert-amyl ether which is then separated to produce the mixed olefin feedstock.

The source of the mixed olefin feedstock is a gas cracking unit or an oxygenate to olefin (OTO) process in one embodiment. Alternatively, a combination of mixed olefins from a gas cracking unit or an OTO process is used. A suitable mixed olefin feedstock contains a mixture of hydrocarbons with an average carbon number of about 4 to 5, 4 to 8, or 4 to 10. These feedstocks can contain 1-olefin, 2-olefin, other internal olefin, and isoolefin as well as saturated hydrocarbons. If the mixed olefin feedstock is from a gas cracking unit, the mixed olefin feedstock will typically contain relatively large amounts of saturated hydrocarbons.

In the preferred embodiment, the source of the olefin feedstock is an OTO process such as an MTO process. One advantage of using olefin produced from an OTO process is the relatively low amounts of isoolefin and saturates in the olefin feedstock stream. For example, olefin produced from an OTO process typically contains from about 70% by weight to about 95% by weight olefin, and less than about 5% by weight isoolefin.

By way of example and not by limitation, the mixed olefin feedstock used in the process can be the C₄, C₄⁺, C₅ or C₅⁺ olefin fraction from an OTO process. The effluent gas removed from an OTO conversion process, particularly a MTO process, typically has a minor amount of hydrocarbons having 4 or more carbon atoms. The amount of hydrocarbons having 4 or more carbon atoms is typically in an amount less than 20 weight percent, based on the total weight of the effluent gas withdrawn from a MTO process, excluding water. In particular with a conversion process of oxygenates into olefin(s) utilizing a molecular sieve catalyst composition the resulting effluent gas typically comprises a majority of ethylene and/or propylene and a minor amount of four carbon and higher carbon number products and other by-products, excluding water.

The C₄⁺ olefin fraction, however, contains greater than 60% by weight, preferably greater than 80% by weight, more preferably greater than 90% by weight, hydrocarbon having four and five carbons. The C₄⁺ olefin fraction contains greater than 50% by weight, preferably greater than 80% by weight, olefin having four carbons. Examples of olefin contained in C₄⁺ olefin fraction are 1-butene, cis and trans 2-butene, isobutene, and the pentenes. The remainder of the C₄⁺ olefin fraction contains paraffin and small amounts of butadiene and other components. The C₄⁺ olefin fraction will more preferably have a compositional range as follows: 70% to 95% by weight, most preferably 80% to 95% by weight, normal butenes, which includes 1-butene and cis and trans 2-butene; 2 to 8% by weight, preferably less than 6% by weight, isobutene; 0.2% to 5% by weight, preferably less than 3% by weight butanes; 2% to 10% by weight, preferably less than 6% by weight, pentenes; and 2% to 10% by weight, preferably less than 5% by weight, propane and propylene.

The C₄⁺ olefin fraction can be used as is, that is, directly from a separation unit of an OTO process to the olefin isomerization unit. Alternatively, there can be some further processing of the C₄⁺ olefin fraction before directing it to the olefin isomerization unit if desired. This optionally includes directing the C₄⁺ olefin fraction to an isoolefin consuming unit, e.g., an etherification process that would selectively convert most if not all of the isobutene to MTBE, and/or to a separation zone to remove a portion of C₅⁺ hydrocarbons.

The preferred catalyst used in the OTO process is a silicoaluminophosphate (SAPO) catalyst. It is preferred that the SAPO molecular sieve used in the OTO process have a relatively low Si/Al₂ ratio. In general, the lower the Si/Al₂ ratio, the lower the C₁-C₄ saturates selectivity, particularly propane selectivity. A Si/Al₂ ratio of less than 0.65 is desirable, with a Si/Al₂ ratio of not greater than 0.40 being preferred, and a Si/Al₂ ratio of not greater than 0.32 being particularly preferred.

The hydrocarbon product from an OTO reaction unit is directed to separation units, known in the art, to separate hydrocarbons according to carbon numbers. For example, methane is separated from the hydrocarbon product followed by, ethylene and ethane (C₂ separation), then propylene and propane (C₃ separation). The remaining portion of the hydrocarbon product, namely the portion containing predominantly four and five carbons (C₄⁺ olefin fraction), is directed to an olefin isomerization unit. Alternatively, the C₄₊ olefin fraction can be separated in the beginning of the separation sequence to reduce the capacity requirements of the C₂/C₃ separation unit by as much as 10% to 25%.

Should additional purification of the mixed olefin feedstock be needed, purification systems such as that found in Kirk-Othmer Encyclopedia of Chemical Technology, 4th edition, Volume 9, John Wiley & Sons, 1996, pg. 894-899, the description of which is incorporated herein by reference, can be used. In addition, purification systems such as that found in Kirk-Othmer Encyclopedia of Chemical Technology, 4th edition, Volume 20, John Wiley & Sons, 1996, pg. 249-271, the description of which is also incorporated herein by reference, can also be used.

The mixed olefin feedstock, in one embodiment, contains one or more diluent(s), typically used to reduce the concentration of the olefin feedstock. The diluent(s) are generally non-reactive to the feedstock or molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. In other embodiments, the feedstock does not contain any diluent. The diluent is used in either a liquid or a vapor form, or a combination thereof.

The diluent is either added directly to a mixed olefin feedstock entering into an isomerization unit or added directly into the isomerization unit, or added with a molecular sieve catalyst composition. In one embodiment, the amount of diluent in the feedstock is in the range of from about 1 to about 99 mole percent based on the total number of moles of the feedstock and diluent, preferably from about 1 to 80 mole percent, more preferably from about 5 to about 50, most preferably from about 5 to about 25. In one embodiment, other hydrocarbons are added to a feedstock either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

In one embodiment of the present invention, the conditions that are effective to isomerize at least a portion of the mixed olefin feedstock to 1-olefin include a temperature greater than 350° C. Additionally or alternatively, the conditions include a pressure and/or weight hour space velocity (WHSV) effective to isomerize at least a portion of a mixed olefin feedstock to form 1-olefin. In another embodiment, the conditions are effective to provide a 1-olefin:isoolefin conversion index, as defined below, of greater than about 1:1. In the process, it is preferred that the 1-olefin:isoolefm conversion index is greater than about 5:1, more preferably greater than about 10:1, and more preferably greater than about 20:1. Ideally, the process will produce a 1-olefin:isoolefin conversion index greater than about 50:1.

The “1-olefin:isoolefin conversion index” is defined as the ratio of 1-olefin to isoolefin produced from the conversion of the mixed olefin feedstock. For example, a mixed olefin feedstock containing 20% by weight 1-butene and 80% by weight 2-butene is converted in an isomerization unit according to the invention to an isomerized product containing 50% by weight 1-butene, 40% by weight 2-butene, and 10% by weight isobutene. The percent conversion of 2-butene in this example is 50%, that is, half of the 2-butenes in the mixed olefin feedstock was converted to isomerized product. Of the amount of 2-butenes converted, 75% was converted to 1-butene and 25% was converted to isobutene. Therefore, the 1-olefin:isoolefin conversion index is 75:25 or 3:1.

The process of isomerizing a mixed olefin feedstock will preferably provide an isomerized product that preferably contains less than 3% by weight isoolefin, more preferably less than 2% by weight isoolefin, and most preferably less than 1% by weight isoolefin. Accordingly, there will be little if any removal of isoolefin from the isomerized olefin product prior to separation of the desired 1-olefin. The process will preferably convert less than 2% by weight, more preferably less than 1% by weight, of the mixed olefin feedstock to a hydrocarbon product containing a higher carbon number.

In accordance with the present invention, catalysts having small pore molecular sieves have proven particularly effective in catalyzing the isomerization of a mixed olefin feedstock to 1-olefin. Molecular sieves are porous solids having pores of different sizes such as zeolites or zeolite-type molecular sieves, carbons and oxides. There are amorphous and crystalline molecular sieves. Molecular sieves include natural, mineral molecular sieves, or chemically formed, synthetic molecular sieves that are typically crystalline materials containing silica, and optionally alumina. The most commercially useful molecular sieves for the petroleum and petrochemical industries are known as zeolites. A zeolite is an aluminosilicate having an open framework structure that usually carries negative charges. This negative charge within portions of the framework is a result of an Al³⁺ replacing a Si⁴⁺. Cations counter-balance these negative charges preserving the electroneutrality of the framework, and these cations are exchangeable with other cations and/or protons. Synthetic molecular sieves, particularly zeolites, are typically synthesized by mixing sources of alumina and silica in a strongly basic aqueous media, often in the presence of a structure directing agent or templating agent. The structure of the molecular sieve formed is determined in part by solubility of the various sources, silica-to-alumina ratio, nature of the cation, synthesis temperature, order of addition, type of templating agent, and the like.

A zeolite is typically formed from corner sharing the oxygen atoms of [SiO₄] and [AlO₄] tetrahedra or octahedra. Zeolites in general have a one-, two- or three-dimensional crystalline pore structure having uniformly sized pores of molecular dimensions that selectively adsorb molecules that can enter the pores, and exclude those molecules that are too large. The pore size, pore shape, interstitial spacing or channels, composition, crystal morphology and structure are a few characteristics of molecular sieves that determine their use in various hydrocarbon adsorption and conversion processes.

Crystalline aluminophosphates, ALPO₄, formed from corner sharing [AlO₂] and [PO₂] tetrahedra linked by shared oxygen atoms are described in U.S. Pat. No. 4,310,440 to produce light olefin(s) from an alcohol. Metal containing aluminophosphate molecular sieves are typically designated as MeAPO's and ElAPO's. MeAPO's have a [MeO₂], [AlO₂] and [PO₂] tetrahedra microporous structure, where Me is a metal source having one or more of the divalent elements Co, Fe, Mg, Mn and Zn, and trivalent Fe from the Periodic Table of Elements. ElAPO's have an [ElO₂], [AlO₂] and [PO₂] tetrahedra microporous structure, where El is a metal source having one or more of the elements As, B, Be, Ga, Ge, Li, Ti and Zr. MeAPO's and ElAPO's are typically synthesized by the hydrothermal crystallization of a reaction mixture of a metal source, an aluminum source, a phosphorous source and a templating agent. The preparation of MeAPO's and ElAPO's are found in U.S. Pat. Nos. 4,310,440, 4,500,651, 4,554,143, 4,567,029, 4,752,651, 4,853,197, 4,873,390 and 5,191,141, which are incorporated herein by reference.

In accordance with the present invention, one of the most useful molecular sieves for isomerizing a mixed olefin feedstock to 1-olefin are those ELAPO's or MeAPO's where the metal source is silicon, often a fumed, colloidal or precipitated silica. These molecular sieves are known as silicoaluminophosphate molecular sieves. Silicoaluminophosphate (SAPO) molecular sieves contain a three-dimensional microporous crystalline framework structure of [SiO₂], [AlO₂] and [PO₂] corner sharing tetrahedral units. SAPO synthesis is described in U.S. Pat. No. 4,440,871, which is herein fully incorporated by reference. SAPO is generally synthesized by the hydrothermal crystallization of a reaction mixture of silicon-, aluminum- and phosphorus-sources and at least one templating agent. Synthesis of a SAPO molecular sieve, its formulation into a SAPO catalyst, and its use in converting a hydrocarbon feedstock into olefin(s), particularly where the feedstock is methanol, is shown in U.S. Pat. Nos. 4,499,327, 4,677,242, 4,677,243, 4,873,390, 5,095,163, 5,714,662 and 6,166,282, all of which are herein fully incorporated by reference.

SAPO molecular sieve catalysts are used in one embodiment of the present invention. A non-limiting example of SAPO catalysts includes: SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, the metal containing forms thereof, and mixtures thereof. As used herein, the term mixture is synonymous with combination and is considered a composition of matter having two or more components in varying proportions, regardless of their physical state.

Small pore SAPO molecular sieve catalysts, such as SAPO-34, are preferably used to catalyze the isomerization of a mixed olefin feedstock to 1-olefin in accordance with the present invention. It is preferred that the silicoaluminophosphate molecular sieve used to isomerize the mixed olefin feedstocks have a Si:Al₂ ratio of less than about 0.33, more preferably less than about 0.25, and most preferably less than about 0.20. In terms of ranges, a Si:Al₂ ratio of from about 0.001 to about 0.33 is preferred, while a Si:Al₂ ratio from about 0.01 to about 0.20 is particularly preferred. The catalyst preferably has a crystal size of less than 2.0 microns, more preferably less than 1.0 microns. The crystal size is preferably greater than 0.05 microns, more preferably greater than 0.1 microns. Thus, the crystal size typically ranges from 0.05 to 2.0 microns, or more preferably, from 0.1 to 1.0 microns. In general, the lower the Si:Al₂ ratio, the greater the product selectivity to 1-olefin.

SAPO molecular sieves are generally classified as being microporous materials having 8, 10, or 12 membered ring structures. These ring structures can have an average pore size ranging from about 3.5-15 angstroms. “Small pore” molecular sieves are defined herein as having an average pore size of less than about 5 angstroms. These pore sizes are typical of molecular sieves having 8 membered rings. “Medium pore” molecular sieve are defined herein as having an average pore size of from about 5 angstroms to about 10 angstroms. “Large pore” molecular sieves are defined herein as having an average pore size of greater than 10 angstroms.

Non-limiting examples of small pore catalysts used in the present invention include: ABW, AEI, AFT, AFX, APC, APD, ATN, ATT, ATV, AWW, BIK, BRE, CAS, CHA, DDR, EAB, EDI, ERI, GIS, GOO, JBW, KFI, LEV, LTA, MER, MON, NAT, PAU, PHI, RHO, RTE, RTH, THO, VNI, YUG, ZON, the substituted forms thereof, and mixtures thereof. Non-limiting examples of small pore SAPO catalysts used in the present invention include: SAPO-17, SAPO-18, SAPO-34, SAPO-35, SAPO-44, the substituted forms thereof, and mixtures thereof. Non-limiting examples of medium pore catalysts used in the present invention include: AEL, AFO, AHT, DAC, EPI, EUO, FER, HEU, LAU, MEL, MFI, MFS, MTT, NES, -PAR, STI, TON, WEI, -WEN, the substituted forms thereof, and mixtures thereof. Non-limiting examples of large pore catalysts used in the present invention include: AFI, AFR, AFS, AFY, ATO, ATS, *BEA, BOG, BPH, CAN, CON, DRO, EMT, FAU, GME, LTL, MAZ, MEI, MOR, MTW, OFF, -RON, VET, the substituted forms thereof, and mixtures thereof.

Small pore catalysts of the non-SAPO variety is used in accordance with one embodiment of the present invention. Non-limiting examples of small pore non-SAPO catalysts useful in the present invention include: CHA, Erionite and Offretite. ZSM-5, ZSM-35 and Ferrierite are particularly preferred medium pore non-SAPO catalysts.

For SAPO-11, platelets having 10-membered rings connecting the faces are preferred. An ECR-42, described in U.S. Pat. No. 6,294,493 B1 to Strohmaier et al., the entirety of which is incorporated herein by reference, is one form of SAPO-11 catalyst which is particularly preferred in accordance with the present invention. The ECR-42 has a disk-like morphology with a thickness of less than about 50 nm, a Si:Al₂ mole ratio of 0.001 to about 0.30, preferably about 0.21, and an alpha value of about 52. The SAPO-11 typically has a crystal size of from about 0.05 microns to about 1.0 microns. This form of catalyst is desirable because it has thin (small) crystals and an excellent Si distribution inside the crystal which leads to the high activity indicated by the alpha value of 52. It is understood by person of ordinary skill in the art that alpha value is determined according to well known procedures inclunding those described in US Patent No which is incorporated herein by reference.

In one embodiment, the molecular sieve is an intergrowth material having two or more distinct phases of crystalline structures within one molecular sieve composition. In particular, intergrowth molecular sieves are described in the U.S. patent application Ser. No. 09/924,016 filed Aug. 7, 2001 and PCT WO 98/15496 published Apr. 16, 1998, both of which are herein fully incorporated by reference. In another embodiment, the molecular sieve comprises at least one intergrown phase of AEI and CHA framework-types. For example, SAPO-1 8, ALPO-18 and RUW-18 have an AEI framework-type, and SAPO-34 has a CHA framework-type.

Metal-substituted SAPOs can also be used in this invention to isomerize a mixed olefin feedstock. These compounds are generally known as MeAPSOs or metal-containing silicoaluminophosphates. The metal can be alkali metals (Group IA), alkaline earth metals (Group IIA), rare earth metals (Group IIIB, including the lanthanide elements), and the transition metals of Groups IB, IIB, IVB, VB, VIB, VIIB, and VIIIB. Incorporation of the metal component is typically accomplished adding the metal component during synthesis of the molecular sieve. However, post-synthesis ion exchange can also be used as disclosed in U.S. Pat. No. 5,962,762 to Sun et al.

Although small pore molecular sieve catalysts are preferred, medium and large pore molecular sieves are used in accordance with the present invention too, particularly at high temperatures.

The silicoaluminophosphate molecular sieves of one embodiment is synthesized by hydrothermal crystallization methods generally known in the art. See, for example, U.S. Pat. Nos. 4,440,871; 4,861,743; 5,096,684; and 5,126,308, the disclosures of which are fully incorporated herein by reference. A reaction mixture is formed by mixing together reactive silicon, aluminum and phosphorus components, along with at least one template. Generally the mixture is sealed and heated, preferably under autogenous pressure, to a temperature of at least 100° C., preferably from 100-250° C., until a crystalline product is formed. Formation of the crystalline product can take anywhere from around 2 hours to as much as 2 weeks. In some cases, stirring or seeding with crystalline material will facilitate the formation of the product.

The silicoaluminophosphate molecular sieve is typically admixed (i.e., blended) with other materials. When blended, the resulting composition is typically referred to as a SAPO catalyst, with the catalyst comprising the SAPO molecular sieve.

Materials which can be blended with the molecular sieve can be various inert or catalytically active materials, or various binder materials. These materials include compositions such as kaolin and other clays, various forms of rare earth metals, metal oxides, other non-zeolite catalyst components, zeolite catalyst components, alumina or alumina sol, titania, zirconia, magnesia, thoria, beryllia, quartz, silica or silica or silica sol, and mixtures thereof. These components are also effective in reducing, inter alia, overall catalyst cost, acting as a thermal sink to assist in heat shielding the catalyst during regeneration, densifying the catalyst and increasing catalyst strength. It is particularly desirable that the inert materials that are used in the catalyst to act as a thermal sink have a heat capacity of from about 0.05 to about 1 cal/g.-° C., more preferably from about 0.1 to about 0.8 cal/g.-° C., most preferably from about 0.1 to about 0.5 cal/g.-° C. The catalyst composition preferably comprises about 10% to about 90%, more preferably about 10% to about 80%, and most preferably about 10% to about 70%, by weight of molecular sieve.

The process for isomerizing a mixed olefin feedstock to 1-olefin in the presence of a molecular sieve catalyst composition of the invention is carried out in a reactor. For example, the process can be a fixed bed process, a fluidized bed process (includes a turbulent bed process), a continuous fluidized bed process, or a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977, which are all herein fully incorporated by reference.

The preferred reactor type are riser reactors generally described in Riser Reactor, Fluidization and Fluid-Particle Systems, pages 48 to 59, F. A. Zenz and D. F. Othmo, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In the preferred embodiment, a fluidized bed process or high velocity fluidized bed process includes a reactor system, a regeneration system and a recovery system.

The reactor system preferably is a fluid bed reactor system having a first reaction zone within one or more riser reactor(s) and a second reaction zone within at least one disengaging vessel, preferably comprising one or more cyclones. In one embodiment, the one or more riser reactor(s) and disengaging vessel is contained within a single reactor vessel. Fresh feedstock, preferably containing a mixture of olefin, optionally with one or more diluent(s), is fed to the one or more riser reactor(s) in which a molecular sieve catalyst composition or coked version thereof is introduced. In one embodiment, the molecular sieve catalyst composition or a coked version thereof is contacted with a liquid or gas, or combination thereof, prior to being introduced to the riser reactor(s), preferably the liquid is water or methanol, and the gas is an inert gas such as nitrogen.

In an embodiment, the amount of fresh feedstock fed separately or jointly with a vapor feedstock, to a reactor system is in the range of from 0.1 weight percent to about 85 weight percent, preferably from about 1 weight percent to about 75 weight percent, more preferably from about 5 weight percent to about 65 weight percent based on the total weight of the feedstock including any diluent contained therein. The liquid and vapor feedstocks are preferably the same composition, or contain varying proportions of the same or different feedstock with the same or different diluent.

The feedstock entering the reactor system is preferably converted, partially or fully, in the first reactor zone into a gaseous effluent that enters the disengaging vessel along with a coked molecular sieve catalyst composition. In the preferred embodiment, cyclone(s) within the disengaging vessel are designed to separate the molecular sieve catalyst composition, preferably a coked molecular sieve catalyst composition, from the gaseous effluent containing one or more olefin(s) within the disengaging zone. Cyclones are preferred, however, gravity effects within the disengaging vessel will also separate the catalyst compositions from the gaseous effluent. Other methods for separating the catalyst compositions from the gaseous effluent include the use of plates, caps, elbows, and the like.

In one embodiment of the disengaging system, the disengaging system includes a disengaging vessel, typically a lower portion of the disengaging vessel is a stripping zone. In the stripping zone the coked molecular sieve catalyst composition is contacted with a gas, preferably one or a combination of steam, methane, carbon dioxide, carbon monoxide, hydrogen, or an inert gas such as argon, preferably steam, to recover adsorbed hydrocarbons from the coked molecular sieve catalyst composition that is then introduced to the regeneration system. In another embodiment, the stripping zone is in a separate vessel from the disengaging vessel and the gas is passed at a gas hourly superficial velocity (GHSV) of from 1 hr⁻¹ to about 20,000 hr⁻¹ based on the volume of gas to volume of coked molecular sieve catalyst composition, preferably at an elevated temperature from 250° C. to about 750° C., preferably from about 350° C. to 650° C., over the coked molecular sieve catalyst composition.

The weight hourly space velocity (WHSV), particularly in a process for converting a feedstock containing a mixture of olefins to 1-olefin in the presence of a molecular sieve catalyst composition within a reaction zone, is defined as the total weight of the feedstock excluding any diluents sent to the reaction zone per hour per weight of molecular sieve in the molecular sieve catalyst composition in the reaction zone. The WHSV preferably is maintained at a level sufficient to keep the catalyst composition in a fluidized state within a reactor.

Typically, the WHSV of the olefin feedstock of one embodiment of the present invention is from about 0.5 hr⁻¹ to about 10,000 hr⁻¹, preferably from about 1 hr⁻¹ to about 1000 hr⁻¹, more preferably from about 1 hr⁻¹ to about 100 hr⁻¹, and more preferably from about 1 hr⁻¹ to about 60 hr⁻¹, and even more preferably from about 1 hr⁻¹ to about 40 hr⁻¹. In one preferred embodiment, the WHSV is greater than about 10 hr⁻¹, 15 hr⁻¹, 20 hr⁻¹ or 25 hr⁻¹. Most preferably, however, the WHSV for conversion of a feedstock containing a mixture of olefin to 1-olefin is in the range of from about 10 hr⁻¹ to about 50 hr⁻¹, about 15 hr⁻to about 50 hr⁻¹, about 20 hr⁻¹ to about 50 hr⁻¹ or about 25 hr⁻¹ to about 50 hr⁻¹.

The superficial gas velocity (SGV) of the feedstock including diluent and reaction products within the reactor system is preferably sufficient to fluidize the molecular sieve catalyst composition within a reaction zone in the reactor. The SGV in the process, particularly within the reactor system, more particularly within the riser reactor(s), is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec. See for example U.S. patent application Ser. No. 09/708,753 filed Nov. 8, 2000, which is herein incorporated by reference.

The method of isomerizing a mixed olefin feedstock to 1-olefin is carried out at relatively high temperatures according to one embodiment of the present invention. The isomerization of mixed olefins to 1-olefin is an equilibrium limited reaction which is driven at high temperatures. The reaction temperature is constrained by equilibrium considerations rather than by catalyst activity. In order to run a process achieving 1-butene reactor yields in excess of 20 percent, isomerization should be carried out about 400° C. Preferably, the mixed olefin feedstock contacts the catalyst at a temperature of at least or greater than 300° C., preferably at least or greater than 350° C., more preferably at least or greater than 400° C., and optionally at least or greater than 500° C. In terms of ranges, the isomerization is carried out at a temperature from about 300° C. to about 700° C., preferably from about 400° C. to about 650° C., more preferably from about 450° C. to about 600° C., and most preferably from about 450° C. to about 550° C. The process of the invention is carried out with the mixed olefin feedstock in the gaseous state.

The partial pressure of the olefin feedstock (excluding any diluent) in the isomerization unit preferably is from about 15 psia to about 500 psia, more preferably from about 15 psia to about 150 psia, and most preferably from about 30 psia to about 100 psia. The total feedstock pressure, including any diluent, in the isomerization unit preferably is less than about 1000 psia and preferably is in the range of about 30-500 psia, more preferably in the range of about 30-400 psia. The above temperatures and pressures can cause carbonaceous deposits or coke to build up in the catalyst pores, thereby rendering the catalysts less effective.

Any coked molecular sieve catalyst composition can be withdrawn from the disengaging vessel, preferably by one or more cyclones(s), and introduced to the regeneration system. The regeneration system comprises a regenerator where the coked catalyst composition is contacted with a regeneration medium, preferably a gas containing oxygen, under general regeneration conditions of temperature, pressure and residence time.

Non-limiting examples of the regeneration medium include one or more of oxygen, O₃, SO₃, N₂O, NO, NO₂, N₂O₅, air, air diluted with nitrogen or carbon dioxide, oxygen and water (U.S. Pat. No. 6,245,703), carbon monoxide and/or hydrogen. The regeneration conditions are those capable of burning coke from the coked catalyst composition, preferably to a level less than 0.5 weight percent based on the total weight of the coked molecular sieve catalyst composition entering the regeneration system. The coked molecular sieve catalyst composition withdrawn from the regenerator forms a regenerated molecular sieve catalyst composition.

The regeneration temperature is in the range of from about 200° C. to about 1500° C., preferably from about 300° C. to about 1000° C., more preferably from about 450° C. to about 750° C., and most preferably from about 500° C. to 700° C. The regeneration pressure is in the range of from about 15 psia (103 kPaa) to about 500 psia (3448 kPaa), preferably from about 20 psia (138 kPaa) to about 250 psia (1724 kPaa), more preferably from about 25 psia (172 kPaa) to about 150 psia (1034 kpaa), and most preferably from about 30 psia (207 kPaa) to about 100 psia (414 kpaa).

The preferred residence time of the molecular sieve catalyst composition in the regenerator is in the range of from about one minute to several hours, most preferably about one minute to 100 minutes, and the preferred volume of oxygen in the gas is in the range of from about 0.01 mole percent to about 5 mole percent based on the total volume of the gas.

In one embodiment, regeneration promoters, typically metal containing compounds such as platinum, palladium and the like, are added to the regenerator directly, or indirectly, for example with the coked catalyst composition. Also, in another embodiment, a fresh molecular sieve catalyst composition is added to the regenerator containing a regeneration medium of oxygen and water as described in U.S. Pat. No. 6,245,703, which is herein fully incorporated by reference.

In an embodiment, a portion of the coked molecular sieve catalyst composition from the regenerator is returned directly to the one or more riser reactor(s), or indirectly, by pre-contacting with the feedstock, or contacting with fresh molecular sieve catalyst composition, or contacting with a regenerated molecular sieve catalyst composition or a cooled regenerated molecular sieve catalyst composition described below.

The burning of coke is an exothermic reaction, and in an embodiment, the temperature within the regeneration system is controlled by various techniques in the art including feeding a cooled gas to the regenerator vessel, operated either in a batch, continuous, or semi-continuous mode, or a combination thereof. A preferred technique involves withdrawing the regenerated molecular sieve catalyst composition from the regeneration system and passing the regenerated molecular sieve catalyst composition through a catalyst cooler that forms a cooled regenerated molecular sieve catalyst composition. The catalyst cooler, in an embodiment, is a heat exchanger that is located either internal or external to the regeneration system.

In one embodiment, the cooler regenerated molecular sieve catalyst composition is returned to the regenerator in a continuous cycle, alternatively, (see U.S. patent application Ser. No. 09/587,766 filed Jun. 6, 2000) a portion of the cooled regenerated molecular sieve catalyst composition is returned to the regenerator vessel in a continuous cycle, and another portion of the cooled molecular sieve regenerated molecular sieve catalyst composition is returned to the riser reactor(s), directly or indirectly, or a portion of the regenerated molecular sieve catalyst composition or cooled regenerated molecular sieve catalyst composition is contacted with by-products within the gaseous effluent (PCT WO 00/49106 published Aug. 24, 2000), which are all herein fully incorporated by reference.

Other methods for operating a regeneration system are in disclosed U.S. Pat. No. 6,290,916 (controlling moisture), which is herein fully incorporated by reference.

The regenerated molecular sieve catalyst composition withdrawn from the regeneration system, preferably from the catalyst cooler, is combined with a fresh molecular sieve catalyst composition and/or re-circulated molecular sieve catalyst composition and/or feedstock and/or fresh gas or liquids, and returned to the riser reactor(s). In another embodiment, the regenerated molecular sieve catalyst composition withdrawn from the regeneration system is returned to the riser reactor(s) directly, preferably after passing through a catalyst cooler. In one embodiment, a carrier, such as an inert gas, feedstock vapor, steam or the like, semi-continuously or continuously, facilitates the introduction of the regenerated molecular sieve catalyst composition to the reactor system, preferably to the one or more riser reactor(s).

By controlling the flow of the regenerated molecular sieve catalyst composition or cooled regenerated molecular sieve catalyst composition from the regeneration system to the reactor system, the optimum level of coke on the molecular sieve catalyst composition entering the reactor is maintained. There are many techniques for controlling the flow of a molecular sieve catalyst composition described in Michael Louge, Experimental Techniques, Circulating Fluidized Beds, Grace, Avidan and Knowlton, eds., Blackie, 1997 (336-337), which is herein incorporated by reference.

Coke levels on the molecular sieve catalyst composition is measured by withdrawing from the conversion process the molecular sieve catalyst composition at a point in the process and determining its carbon content. Typical levels of coke on the molecular sieve catalyst composition, after regeneration is in the range of from 0.01 weight percent to about 15 weight percent, preferably from about 0.1 weight percent to about 10 weight percent, more preferably from about 0.2 weight percent to about 5 weight percent, and most preferably from about 0.3 weight percent to about 2 weight percent based on the total weight of the molecular sieve and not the total weight of the molecular sieve catalyst composition.

In one preferred embodiment, the mixture of fresh molecular sieve catalyst composition and regenerated molecular sieve catalyst composition and/or cooled regenerated molecular sieve catalyst composition contains in the range of from about 1 to 50 weight percent, preferably from about 2 to 30 weight percent, more preferably from about 2 to about 20 weight percent, and most preferably from about 2 to about 10 coke or carbonaceous deposit based on the total weight of the mixture of molecular sieve catalyst compositions. See for example U.S. Pat. No. 6,023,005, which is herein fully incorporated by reference.

The at least partially isomerized effluent stream that exits the isomerization unit includes more 1-olefin than the olefin feedstock that was introduced into the isomerization unit. Preferably, the effluent includes more than about 10 or 20 weight percent 1-olefin. More preferably, the effluent includes more than about 20 or 25 weight percent 1-olefin. Most preferably, the effluent includes more than about 25 or 35 weight percent 1-olefin. In terms of ranges, the at least partially isomerized effluent stream can contain from about 10-50 weight percent 1-olefin, more preferably from about 20-50 weight percent 1-olefin, and most preferably from about 30-50 weight percent 1-olefin. The composition of the isomerized effluent, of one embodiment, is characterized in terms of percent increase in 1-olefin. In one example, the effluent contains about 10 or 20 weight percent, more preferably about 20 or 25 weight percent, and most preferably about 35 weight percent more 1-olefin than was in the olefin feedstock stream. In terms of ranges, the effluent preferably contains from about 10-35 percent, more preferably from about 20-35 percent, and more preferably from 30-35 percent more 1-olefin than was in the olefin feedstock stream. In other words, the isomerization process can result in a 10, 20, 30 or 35 percent increase in 1-olefin concentration. The effluent will also include unisomerized olefin such as internal olefin, e.g., 2-butene. The effluent can also include isoolefin and diene, the processing of which is discussed in more detail below. Preferably, at least a portion of the unisomerized olefin is recycled back to the isomerization unit for further isomerization to 1-olefin. Optionally, the unisomerized olefin is directed to and combined with the feedstock stream prior to its introduction to the isomerization unit. Additionally or alternatively, the isoolefin and/or diene in the effluent is removed, as discussed below. The effluent sometimes includes a minor amount of inert compounds such as paraffins, which preferably are periodically or continuously removed from the reaction system through a purge stream.

The present invention provides for high 1-butene selectivities. For example, selectivities greater than about 70 percent are easily obtainable. Preferably, the selectivity is greater than about 80 or 90 percent. Selectivities as high as or greater than about 95, 96, 97, 98, and even 99 percent are readily obtainable. In terms of ranges, the 1-olefin selectivity is from about 70-100 percent, preferably from about 80-100 percent, more preferably from 90-100 percent, more preferably from 95-100 percent, and most preferably from 97-100 percent. The present invention also provides for very low isoolefin selectivity, preferably below about 5.0 weight percent, more preferably below about 3.0, 1.0, 0.5 or 0.1 weight percent. The isoolefin content in the isomerized stream is undetectable in some instances. In terms of ranges, the isoolefin selectivity is from about 0 to about 5.0 weight percent, more preferably from about 0 to about 3.0 weight percent, and most preferably from about 0 to about 1.0 weight percent. It is believed that the shape selectivity of small pore molecular sieve catalysts such as SAPO-34 reduces or eliminates the formation of isobutene. Any isobutene formed diffuses slowly through the catalyst cages. Isobutene can isomerize under the isomerization conditions to linear butenes which rapidly escape the pores of the catalyst.

An isoolefin removal unit such as an etherification unit can be used to convert the small amounts of isobutene produced in the isomerization process to additional alkyl ether. Alternatively, the isoolefin produced is recycled to an isoolefin removal unit which is upstream of the isomerization unit. However, because of the relatively high 1-olefin:isoolefin conversion index of the present invention, recycling and/or secondary isoolefin removal units are generally not required.

Similarly, a diene removal unit such as a diene hydrofiner can be used to convert the minor amounts of diene produced in the isomerization process to C₄ monoolefin. Alternatively, the diene produced is recycled to a diene removal unit which is upstream of the isomerization unit. However, depending on the reaction conditions in the isomerization unit, recycling of diene and/or directing the diene to a diene removal unit between the isomerization unit and the separation unit will likely be necessary, as disclosed below in reference to the figures.

1-Olefin that has been formed in the catalyzed isomerization process of the present invention is directed with unisomerized olefin (non-1-olefin) to a separation and purification system. The gaseous effluent is withdrawn from the disengaging system and is passed through a recovery system. There are many well known recovery systems, techniques and sequences that are useful in separating olefin(s) and purifying olefin(s) from the gaseous effluent. Recovery systems generally comprise one or more or a combination of a various separation, fractionation and/or distillation towers, columns, splitters, or trains, reaction systems such as ethylbenzene manufacture (U.S. Pat. No. 5,476,978) and other derivative processes such as aldehydes, ketones and ester manufacture (U.S. Pat. No. 5,675,041), and other associated equipment for example various condensers, heat exchangers, refrigeration systems or chill trains, compressors, knock-out drums or pots, pumps, and the like.

Non-limiting examples of these towers, columns, splitters or trains used alone or in combination include one or more of a demethanizer, preferably a high temperature demethanizer, a dethanizer, a depropanizer, preferably a wet depropanizer, a wash tower often referred to as a caustic wash tower and/or quench tower, absorbers, adsorbers, membranes, ethylene (C2) splitter, propylene (C3) splitter, butene (C4) splitter, and the like.

Various recovery systems useful for recovering olefin(s) are described in U.S. Pat. No. 5,960,643 (secondary rich ethylene stream), U.S. Pat. Nos. 5,019,143, 5,452,581 and 5,082,481 (membrane separations), U.S. Pat. No. 5,672,197 (pressure dependent adsorbents), U.S. Pat. No. 6,069,288 (hydrogen removal), U.S. Pat. No. 5,904,880 (recovered methanol to hydrogen and carbon dioxide in one step), U.S. Pat. No. 5,927,063 (recovered methanol to gas turbine power plant), and U.S. Pat. No. 6,121,504 (direct product quench), U.S. Pat. No. 6,121,503 (high purity olefins without superfractionation), and U.S. Pat. No. 6,293,998 (pressure swing adsorption), which are all herein fully incorporated by reference.

Other recovery systems that include purification systems, for example for the purification of olefin(s), are described in Kirk-Othmer Encyclopedia of Chemical Technology, 4th Edition, Volume 9, John Wiley & Sons, 1996, pages 249-271 and 894-899, which is herein incorporated by reference. Purification systems are also described in for example, U.S. Pat. No. 6,271,428 (purification of a diolefin hydrocarbon stream), U.S. Pat. No. 6,293,999 (separating propylene from propane), and U.S. patent application Ser. No. 09/689,363 filed Oct. 20, 2000 (purge stream using hydrating catalyst), which is herein incorporated by reference.

Preferably, the majority of 1-olefin is separated from the at least partially isomerized effluent through an overhead or top product stream in a separation unit. The top product stream can include a minor amount of isoolefin, which likewize can be removed from the top product stream through an isoolefin removal unit. The majority of the unisomerized olefin, the majority of which preferably is internal olefin, is separated from the effluent through a bottoms stream. The bottoms stream preferably is redirected to the isomerization unit. Optionally, as discussed above, the unisomerized olefin can pass through a diene removal unit, and/or an isoolefin removal unit prior to being recycled to the isomerization unit. Inert compounds can be removed from the bottoms stream before it is recycled to the isomerization unit through a purge stream. The inert compounds, e.g., n-butane, isobutane or other paraffins, may have been formed in the OTO process, the diene removal process, the isoolefin removal process, and/or the isomerization process.

As indicated above, the present invention achieves the isomerization to 1-olefin preferably by implementing a small pore molecular sieve catalyst such as SAPO-34. Catalysts of this variety can also be implemented in the OTO process. Accordingly, the catalysts used in the present isomerization invention can be supplied from or to an OTO process. As the characteristics of the catalyst in the OTO process changes, at least a portion of the OTO catalyst can be directed to the isomerization unit if the OTO catalyst maintains properties conducive to the formation of 1-olefin from a mixed olefin feedstock. Similarly, as the characteristics of the catalyst in the isomerization unit changes, at least a portion of the isomerization catalyst can be directed to the OTO unit to participate in the oxygenate to olefin reaction if the isomerization catalyst maintains properties conducive to the formation of olefins from oxygenates. In this manner, reaction efficiency can be maximized in both the OTO reactor system and in the isomerization reactor system. Additionally or alternatively, the catalyst in the isomerization reactor system can be periodically stripped and/or regenerated and directed back to the isomerization reactor system and/or to the OTO reactor system. Similarly, the catalyst in the OTO reactor system can be periodically stripped and/or regenerated and directed back to the OTO unit and/or to the isomerization reactor system. One OTO reactor of the present invention is discussed in more detail below.

In a preferred embodiment, the OTO reactor system is incorporated with the isomerization system and comprises an oxygenate feedstock containing one or more oxygenates, more specifically, one or more organic compound(s) containing at least one oxygen atom is converted preferably to ethylene and/or propylene. In the most preferred embodiment of the process of the present invention, the oxygenate in the feedstock is one or more alcohol(s), preferably aliphatic alcohol(s) where the aliphatic moiety of the alcohol(s) has from 1 to 20 carbon atoms, preferably from 1 to 10 carbon atoms, and most preferably from 1 to 4 carbon atoms. The alcohols useful as feedstock in the process of the invention include lower straight and branched chain aliphatic alcohols and their unsaturated counterparts. Non-limiting examples of oxygenates include methanol, ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof. In the most preferred embodiment, the feedstock is selected from one or more of methanol, ethanol, dimethyl ether, diethyl ether or a combination thereof, more preferably methanol and dimethyl ether, and most preferably methanol.

The feedstock of the OTO system, in one embodiment, contains one or more diluent(s), typically used to reduce the concentration of the feedstock, and are generally non-reactive to the feedstock or molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred.

The diluent is either added directly to a feedstock entering into a reactor or added directly into a reactor, or added with a molecular sieve catalyst composition. In one embodiment, the amount of diluent in the feedstock is in the range of from about 1 to about 99 mole percent based on the total number of moles of the feedstock and diluent, preferably from about 1 to 80 mole percent, more preferably from about 5 to about 50, most preferably from about 5 to about 25. In one embodiment, other hydrocarbons are added to a feedstock either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

Molecular sieves capable of converting an oxygenate to an olefin compound include zeolite as well as non-zeolite molecular sieves, and are of the large, medium or small pore type. Non-limiting examples of these molecular sieves are the small pore molecular sieves, AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof; the medium pore molecular sieves, AFO, AEL, EUO, HEU, FER, MEL, MFI, MTW, MTT, TON, and substituted forms thereof, and the large pore molecular sieves, EMT, FAU, and substituted forms thereof. Other molecular sieves include ANA, BEA, CFI, CLO, DON, GIS, LTL, MER, MOR, MWW and SOD. Non-limiting examples of the preferred molecular sieves, particularly for converting an oxygenate containing feedstock into olefin(s), include AEL, AFY, BEA, CHA, EDI, FAU, FER, GIS, LTA, LTL, MER, MFI, MOR, MTT, MWW, TAM and TON. In one preferred embodiment, the molecular sieve of the invention has an AEI topology or a CHA topology, or a combination thereof, most preferably a CHA topology.

Molecular sieve materials all have 3-dimensional, four-connected framework structure of corner-sharing TO₄ tetrahedra, where T is any tetrahedrally coordinated cation. These molecular sieves are typically described in terms of the size of the ring that defines a pore, where the size is based on the number of T atoms in the ring. Other framework-type characteristics include the arrangement of rings that form a cage, and when present, the dimension of channels, and the spaces between the cages. See van Bekkum, et al., Introduction to Zeolite Science and Practice, Second Completely Revised and Expanded Edition, Volume 137, pages 1-67, Elsevier Science, B.V., Amsterdam, Netherlands (2001).

The small, medium and large pore molecular sieves have from a 4-ring to a 12-ring or greater framework-type. In a preferred embodiment of the OTO process, the molecular sieves have 8-, 10- or 12- ring structures or larger and an average pore size in the range of from about 3 Å to 15 Å. In the most preferred embodiment, the molecular sieves of the invention, preferably silicoaluminophosphate molecular sieves, have 8-rings and an average pore size less than about 5 Å, preferably in the range of from 3 Å to about 5 Å, more preferably from 3 Å to about 4.5 Å, and most preferably from 3.5 Å to about 4.2 Å.

Molecular sieves, particularly zeolitic and zeolitic-type molecular sieves, preferably have a molecular framework of one, preferably two or more corner-sharing [TO₄] tetrahedral units, more preferably, two or more [SiO₄], [AlO₄] and/or [PO₄] tetrahedral units, and most preferably [SiO₄], [AlO₄] and [PO₄] tetrahedral units. These silicon, aluminum, and phosphorous based molecular sieves and metal containing silicon, aluminum and phosphorous based molecular sieves have been described in detail in numerous publications including for example, U.S. Pat. No. 4,567,029 (MeAPO where Me is Mg, Mn, Zn, or Co), U.S. Pat. No. 4,440,871 (SAPO), European Patent Application EP-A-0 159 624 (ELAPSO where El is As, Be, B, Cr, Co, Ga, Ge, Fe, Li, Mg, Mn, Ti or Zn), U.S. Pat. No. 4,554,143 (FeAPO), U.S. Pat. Nos. 4,822,478, 4,683,217, 4,744,885 (FeAPSO), EP-A-0 158 975 and U.S. Pat. No. 4,935,216 (ZnAPSO, EP-A-0 161 489 (CoAPSO), EP-A-0 158 976 (ELAPO, where EL is Co, Fe, Mg, Mn, Ti or Zn), U.S. Pat. No. 4,310,440 (AlPO₄), EP-A-0 158 350 (SENAPSO), U.S. Pat. No. 4,973,460 (LiAPSO), U.S. Pat. No. 4,789,535 (LiAPO), U.S. Pat. No. 4,992,250 (GeAPSO), U.S. Pat. No. 4,888,167 (GeAPO), U.S. Pat. No. 5,057,295 (BAPSO), U.S. Pat. No. 4,738,837 (CrAPSO), U.S. Pat. Nos. 4,759,919, and 4,851,106 (CrAPO), U.S. Pat. Nos. 4,758,419, 4,882,038, 5,434,326 and 5,478,787 (MgAPSO), U.S. Pat. No. 4,554,143 (FeAPO), U.S. Pat. No. 4,894,213 (AsAPSO), U.S. Pat. No. 4,913,888 (AsAPO), U.S. Pat. Nos. 4,686,092, 4,846,956 and 4,793,833 (MnAPSO), U.S. Pat. Nos. 5,345,011 and 6,156,931 (MnAPO), U.S. Pat. No. 4,737,353 (BeAPSO), U.S. Pat. No. 4,940,570 (BeAPO), U.S. Pat. Nos. 4,801,309, 4,684,617 and 4,880,520 (TiAPSO), U.S. Pat. Nos. 4,500,651, 4,551,236 and 4,605,492 (TiAPO), U.S. Pat. Nos. 4,824,554, 4,744,970 (CoAPSO), U.S. Pat. No. 4,735,806 (GaAPSO) EP-A-0 293 937 (QAPSO, where Q is framework oxide unit [QO₂]), as well as U.S. Pat. Nos. 4,567,029, 4,686,093, 4,781,814, 4,793,984, 4,801,364, 4,853,197, 4,917,876, 4,952,384, 4,956,164, 4,956,165, 4,973,785, 5,241,093, 5,493,066 and 5,675,050, all of which are herein fully incorporated by reference.

Other molecular sieves include those described in EP-0 888 187 B1 (microporous crystalline metallophosphates, SAPO₄ (UIO-6)), U.S. Pat. No. 6,004,898 (molecular sieve and an alkaline earth metal), U.S. patent application Ser. No. 09/511,943 filed Feb. 24, 2000 (integrated hydrocarbon co-catalyst), PCT WO 01/64340 published Sep. 7, 2001(thorium containing molecular sieve), and R. Szostak, Handbook of Molecular Sieves, Van Nostrand Reinhold, New York, N.Y. (1992), which are all herein fully incorporated by reference.

The more preferred silicon, aluminum and/or phosphorous containing molecular sieves, and aluminum, phosphorous, and optionally silicon, containing molecular sieves include aluminophosphate (ALPO) molecular sieves and silicoaluminophosphate (SAPO) molecular sieves and substituted, preferably metal substituted, ALPO and SAPO molecular sieves. The most preferred molecular sieves are SAPO molecular sieves, and metal substituted SAPO molecular sieves. In an embodiment, the metal is an alkali metal of Group IA of the Periodic Table of Elements, an alkaline earth metal of Group IIA of the Periodic Table of Elements, a rare earth metal of Group IIIB, including the Lanthanides: lanthanum, cerium, praseodymium, neodymium, samarium, europium, gadolinium, terbium, dysprosium, holmium, erbium, thulium, ytterbium and lutetium; and scandium or yttrium of the Periodic Table of Elements, a transition metal of Groups IVB, VB, VIB, VIIB, VIIIB, and IB of the Periodic Table of Elements, or mixtures of any of these metal species. In one preferred embodiment, the metal is selected from the group consisting of Co, Cr, Cu, Fe, Ga, Ge, Mg, Mn, Ni, Sn, Ti, Zn and Zr, and mixtures thereof. In another preferred embodiment, these metal atoms discussed above are inserted into the framework of a molecular sieve through a tetrahedral unit, such as [MeO₂], and carry a net charge depending on the valence state of the metal substituent. For example, in one embodiment, when the metal substituent has a valence state of +2, +3, +4, +5, or +6, the net charge of the tetrahedral unit is between −2 and +2.

In one embodiment, the molecular sieve, as described in many of the U.S. Patents mentioned above, is represented by the empirical formula, on an anhydrous basis:

mR:(MₓAl_{y}P_{z})O₂

wherein R represents at least one templating agent, preferably an organic templating agent; m is the number of moles of R per mole of (MₓAl_{y}P_{z})O₂ and m has a value from 0 to 1, preferably 0 to 0.5, and most preferably from 0 to 0.3; x, y, and z represent the mole fraction of Al, P and M as tetrahedral oxides, where M is a metal selected from one of Group IA, IIA, IB, IIIB, IVB, VB, VIB, VIIB, VIIIB and Lanthanide's of the Periodic Table of Elements, preferably M is selected from one of the group consisting of Co, Cr, Cu, Fe, Ga, Ge, Mg, Mn, Ni, Sn, Ti, Zn and Zr. In an embodiment, m is greater than or equal to 0.2, and x, y and z are greater than or equal to 0.01.

In another embodiment, m is greater than 0.1 to about 1, x is greater than 0 to about 0.25, y is in the range of from 0.4 to 0.5, and z is in the range of from 0.25 to 0.5, more preferably m is from 0.15 to 0.7, x is from 0.01 to 0.2, y is from 0.4 to 0.5, and z is from 0.3 to 0.5.

Non-limiting examples of SAPO and ALPO molecular sieves used in the OTO process which include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44 (U.S. Pat. No. 6,162,415), SAPO-47, SAPO-56, ALPO-5, ALPO-11, ALPO-18, ALPO-31, ALPO-34, ALPO-36, ALPO-37, ALPO-46, and metal containing molecular sieves thereof. The more preferred zeolite-type molecular sieves include one or a combination of SAPO-18, SAPO-34, SAPO-35, SAPO-44, SAPO-56, ALPO-18 and ALPO-34, even more preferably one or a combination of SAPO-18, SAPO-34, ALPO-34 and ALPO-18, and metal containing molecular sieves thereof, and most preferably one or a combination of SAPO-34 and ALPO-18, and metal containing molecular sieves thereof.

In one embodiment of the OTO reactor system, the molecular sieve is an intergrowth material having two or more distinct phases of crystalline structures within one molecular sieve composition. In particular, intergrowth molecular sieves are described in the U.S. patent application Ser. No. 09/924,016 filed Aug. 7, 2001 and PCT WO 98/15496 published Apr. 16, 1998, both of which are herein fully incorporated by reference. In another embodiment, the molecular sieve comprises at least one intergrown phase of AEI and CHA framework-types. For example, SAPO-18, ALPO-18 and RUW-18 have an AEI framework-type, and SAPO-34 has a CHA framework-type.

In another embodiment, the molecular sieves used in the invention are combined with one or more other molecular sieves. In another embodiment, the preferred silicoaluminophosphate or aluminophosphate molecular sieves, or a combination thereof, are combined with one more of the following non-limiting examples of molecular sieves described in the following: Beta (U.S. Pat. No. 3,308,069), ZSM-5 (U.S. Pat. Nos. 3,702,886, 4,797,267 and 5,783,321), ZSM-11 (U.S. Pat. No. 3,709,979), ZSM-12 (U.S. Pat. No. 3,832,449), ZSM-12 and ZSM-38 (U.S. Pat. No. 3,948,758), ZSM-22 (U.S. Pat. No. 5,336,478), ZSM-23 (U.S. Pat. No. 4,076,842), ZSM-34 (U.S. Pat. No. 4,086,186), ZSM-35 (U.S. Pat. No. 4,016,245, ZSM-48 (U.S. Pat. No. 4,397,827), ZSM-58 (U.S. Pat. No. 4,698,217), MCM-1 (U.S. Pat. No. 4,639,358), MCM-2 (U.S. Pat. No. 4,673,559), MCM-3 (U.S. Pat. No. 4,632,811), MCM-4 (U.S. Pat. No. 4,664,897), MCM-5 (U.S. Pat. No. 4,639,357), MCM-9 (U.S. Pat. No. 4,880,611), MCM-10 (U.S. Pat. No. 4,623,527), MCM-14 (U.S. Pat. No. 4,619,818), MCM-22 (U.S. Pat. No. 4,954,325), MCM-41 (U.S. Pat. No. 5,098,684), M-41S (U.S. Pat. No. 5,102,643), MCM-48 (U.S. Pat. No. 5,198,203), MCM-49 (U.S. Pat. No. 5,236,575), MCM-56 (U.S. Pat. No. 5,362,697), ALPO-11 (U.S. Pat. No. 4,310,440), titanium aluminosilicates (TASO), TASO-45 (EP-A-0 229,-295), boron silicates (U.S. Pat. No. 4,254,297), titanium aluminophosphates (TAPO) (U.S. Pat. No. 4,500,651), mixtures of ZSM-5 and ZSM-11 (U.S. Pat. No. 4,229,424), ECR-18 (U.S. Pat. No. 5,278,345), SAPO-34 bound ALPO-5 (U.S. Pat. No. 5,972,203), PCT WO 98/57743 published Dec. 23, 1988 (molecular sieve and Fischer-Tropsch), U.S. Pat. No. 6,300,535 (MFI-bound zeolites), and mesoporous molecular sieves (U.S. Pat. Nos. 6,284,696, 5,098,684, 5,102,643 and 5,108,725), which are all herein fully incorporated by reference.

The molecular sieves are made or formulated into catalysts by combining the synthesized molecular sieves with a binder and/or a matrix material to form a molecular sieve catalyst composition or a formulated molecular sieve catalyst composition. This formulated molecular sieve catalyst composition is formed into useful shape and sized particles by conventional techniques such as spray drying, pelletizing, extrusion, and the like.

There are many different binders that are useful in forming the molecular sieve catalyst composition. Non-limiting examples of binders that are useful alone or in combination include various types of hydrated alumina, silicas, and/or other inorganic oxide sol. One preferred alumina containing sol is aluminum chlorhydrol. The inorganic oxide sol acts like glue binding the synthesized molecular sieves and other materials such as the matrix together, particularly after thermal treatment. Upon heating, the inorganic oxide sol, preferably having a low viscosity, is converted into an inorganic oxide matrix component. For example, an alumina sol will convert to an aluminum oxide matrix following heat treatment.

Aluminum chlorhydrol, a hydroxylated aluminum based sol containing a chloride counter ion, has the general formula of AlₘOₙ(OH)ₒClₚ.x(H₂O) wherein m is 1 to 20, n is 1 to 8, o is 5 to 40, p is 2 to 15, and x is 0 to 30. In one embodiment, the binder is Al₁₃O₄(OH)₂₄Cl₇.12(H₂O) as is described in G. M. Wolterman, et al., Stud. Surf. Sci. and Catal., 76, pages 105-144 (1993), which is herein incorporated by reference. In another embodiment, one or more binders are combined with one or more other non-limiting examples of alumina materials such as aluminum oxyhydroxide, γ-alumina, Boehmite, diaspore, and transitional aluminas such as α-alumina, β-alumina, γ-alumina, δ-alumina, ε-alumina, κ-alumina, and ρ-alumina, aluminum trihydroxide, such as Gibbsite, Bayerite, Nordstrandite, Doyelite, and mixtures thereof.

In another embodiment, the binders are alumina sols, predominantly comprising aluminum oxide, optionally including some silicon. In yet another embodiment, the binders are peptized alumina made by treating alumina hydrates such as Pseudobohemite, with an acid, preferably an acid that does not contain a halogen, to prepare sols or aluminum ion solutions. Non-limiting examples of commercially available colloidal alumina sols include Nalco 8676 available from Nalco Chemical Co., Naperville, Ill., and Nyacol available from The PQ Corporation, Valley Forge, Pa.

The molecular sieve, in a preferred embodiment, is combined with one or more matrix material(s). Matrix materials are typically effective in reducing overall catalyst cost, act as thermal sinks assisting in shielding heat from the catalyst composition for example during regeneration, densifying the catalyst composition, increasing catalyst strength such as crush strength and attrition resistance, and to control the rate of conversion in a particular process.

Non-limiting examples of matrix materials include one or more of: rare earth metals, metal oxides including titania, zirconia, magnesia, thoria, beryllia, quartz, silica or sols, and mixtures thereof, for example silica-magnesia, silica-zirconia, silica-titania, silica-alumina and silica-alumina-thoria. In an embodiment, matrix materials are natural clays such as those from the families of Montmorillonite and kaolin. These natural clays include Sabbentonites and those kaolins known as, for example, Dixie, McNamee, Georgia and Florida clays. Non-limiting examples of other matrix materials include: Haloysite, Kaolinite, Dickite, Nacrite, or Anauxite. In one embodiment, the matrix material, preferably any of the clays, are subjected to well known modification processes such as calcination and/or acid treatment and/or chemical treatment.

In one preferred embodiment, the matrix material is a clay or a clay-type composition, preferably the clay or clay-type composition having a low iron or titania content, and most preferably the matrix material is kaolin. Kaolin has been found to form a pumpable, high solid content slurry, it has a low fresh surface area, and it packs together easily due to its platelet structure. A preferred average particle size of the matrix material, most preferably kaolin, is from about 0.1 μm to about 0.6 μm with a D90 particle size distribution of less than about 1 μm.

In another embodiment, the weight ratio of the binder to the matrix material used in the formation of the molecular sieve catalyst composition is from 0:1 to 1:15, preferably 1:15 to 1:5, more preferably 1:10 to 1:4, and most preferably 1:6 to 1:5. It has been found that a higher sieve content, lower matrix content, increases the molecular sieve catalyst composition performance, however, lower sieve content, higher matrix material, improves the attrition resistance of the composition.

In another embodiment, the formulated molecular sieve catalyst composition contains from about 1% to about 99%, more preferably from about 5% to about 90%, and most preferably from about 10% to about 80%, by weight of the molecular sieve based on the total weight of the molecular sieve catalyst composition.

In another embodiment, the weight percent of binder in or on the spray dried molecular sieve catalyst composition based on the total weight of the binder, molecular sieve, and matrix material is from about 2% by weight to about 30% by weight, preferably from about 5% by weight to about 20% by weight, and more preferably from about 7% by weight to about 15% by weight.

Once the molecular sieve catalyst composition is formed in a substantially dry or dried state, to further harden and/or activate the formed catalyst composition, a heat treatment such as calcination, at an elevated temperature is usually performed. A conventional calcination environment is air that typically includes a small amount of water vapor. Typical calcination temperatures are in the range from about 400° C. to about 1,000° C., preferably from about 500° C. to about 800° C., and most preferably from about 550° C. to about 700° C., preferably in a calcination environment such as air, nitrogen, helium, flue gas (combustion product lean in oxygen), or any combination thereof.

The OTO process for converting a feedstock, especially a feedstock containing one or more oxygenates, in the presence of a molecular sieve catalyst composition of the invention, is carried out in a reaction process in a reactor, where the process is a fixed bed process, a fluidized bed process (includes a turbulent bed process), preferably a continuous fluidized bed process, and most preferably a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977, which are all herein fully incorporated by reference.

The preferred reactor type are riser reactors generally described in Riser Reactor, Fluidization and Fluid-Particle Systems, pages 48 to 59, F. A. Zenz and D. F. Othmo, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In the preferred embodiment, a fluidized bed process or high velocity fluidized bed process includes a reactor system, a regeneration system and a recovery system. The reactor system preferably is a fluid bed reactor system having a first reaction zone within one or more riser reactor(s) and a second reaction zone within at least one disengaging vessel, preferably comprising one or more cyclones. In one embodiment, the one or more riser reactor(s) and disengaging vessel is contained within a single reactor vessel. Fresh feedstock, preferably containing one or more oxygenates, optionally with one or more diluent(s), is fed to the one or more riser reactor(s) in which a zeolite or zeolite-type molecular sieve catalyst composition or coked version thereof is introduced. In one embodiment, the molecular sieve catalyst composition or coked version thereof is contacted with a liquid or gas, or combination thereof, prior to being introduced to the riser reactor(s), preferably the liquid is water or methanol, and the gas is an inert gas such as nitrogen.\

In an OTO system embodiment, the amount of fresh oxygenate feedstock fed separately or jointly with a vapor feedstock, to a reactor system is in the range of from 0.1 weight percent to about 85 weight percent, preferably from about 1 weight percent to about 75 weight percent, more preferably from about 5 weight percent to about 65 weight percent based on the total weight of the feedstock including any diluent contained therein. The liquid and vapor feedstocks are preferably the same composition, or contain varying proportions of the same or different feedstock with the same or different diluent.

The feedstock entering the OTO reactor system is preferably converted, partially or fully, in the first reactor zone into a gaseous effluent that enters the disengaging vessel along with a coked molecular sieve catalyst composition. In the preferred embodiment, cyclone(s) within the disengaging vessel are designed to separate the molecular sieve catalyst composition, preferably a coked molecular sieve catalyst composition, from the gaseous effluent containing one or more olefin(s) within the disengaging zone. Cyclones are preferred, however, gravity effects within the disengaging vessel will also separate the catalyst compositions from the gaseous effluent. Other methods for separating the catalyst compositions from the gaseous effluent include the use of plates, caps, elbows, and the like.

In one embodiment of the OTO disengaging system, the disengaging system includes a disengaging vessel, typically a lower portion of the disengaging vessel is a stripping zone. In the stripping zone the coked molecular sieve catalyst composition is contacted with a gas, preferably one or a combination of steam, methane, carbon dioxide, carbon monoxide, hydrogen, or an inert gas such as argon, preferably steam, to recover adsorbed hydrocarbons from the coked molecular sieve catalyst composition that is then introduced to the regeneration system. In another embodiment, the stripping zone is in a separate vessel from the disengaging vessel and the gas is passed at a gas hourly superficial velocity (GHSV) of from 1 hr⁻¹ to about 20,000 hr⁻¹ based on the volume of gas to volume of coked molecular sieve catalyst composition, preferably at an elevated temperature from 250° C. to about 750° C., preferably from about 350° C. to 650° C., over the coked molecular sieve catalyst composition.

The conversion temperature employed in the OTO conversion process, specifically within the reactor system, is in the range of from about 200° C. to about 1,000° C., preferably from about 250° C. to about 800° C., more preferably from about 250° C. to about 750° C., yet more preferably from about 300° C. to about 650° C., yet even more preferably from about 350° C. to about 600° C. most preferably from about 350° C. to about 550° C.

The conversion pressure employed in the conversion process, specifically within the reactor system, is not critical. The conversion pressure is based on the partial pressure of the feedstock exclusive of any diluent therein. Typically the conversion pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), particularly in an OTO process for converting a feedstock containing one or more oxygenates in the presence of a molecular sieve catalyst composition within a reaction zone, is defined as the total weight of the feedstock excluding any diluents to the reaction zone per hour per weight of molecular sieve in the molecular sieve catalyst composition in the reaction zone. The WHSV is maintained at a level sufficient to keep the catalyst composition in a fluidized state within a reactor.

Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing methanol and dimethyl ether is in the range of from about 20 hr⁻¹ to about 300 hr⁻¹.

The superficial gas velocity (SGV) of the feedstock including diluent and reaction products within the OTO reactor system is preferably sufficient to fluidize the molecular sieve catalyst composition within a reaction zone in the reactor. The SGV in the process, particularly within the reactor system, more particularly within the riser reactor(s), is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec. See for example U.S. patent application Ser. No. 09/708,753 filed Nov. 8, 2000, which is herein incorporated by reference.

In one preferred embodiment of the process for converting an oxygenate to olefin(s) using a silicoaluminophosphate molecular sieve catalyst composition, the process is operated at a WHSV of at least 20 hr⁻¹ and a Temperature Corrected Normalized Methane Selectivity (TCNMS) of less than 0.016, preferably less than or equal to 0.01. See for example U.S. Pat. No. 5,952,538, which is herein fully incorporated by reference.

In another embodiment of the processes for converting an oxygenate such as methanol to one or more olefin(s) using a molecular sieve catalyst composition, the WHSV is from 0.01 hr⁻¹ to about 100 hr⁻¹, at a temperature of from about 350° C. to 550° C., and silica to Me₂O₃ (Me is a Group IIIA or VIII element from the Periodic Table of Elements) molar ratio of from 300 to 2500. See for example EP-0 642 485 B1, which is herein fully incorporated by reference.

Other processes for converting an oxygenate such as methanol to one or more olefin(s) using a molecular sieve catalyst composition are described in PCT WO 01/23500 published Apr. 5, 2001 (propane reduction at an average catalyst feedstock exposure of at least 1.0), which is herein incorporated by reference.

According to one embodiment, the conversion of the primary oxygenate, e.g., methanol, is from 90 wt % to 98 wt %. According to another embodiment the conversion of methanol is from 92 wt % to 98 wt %, preferably from 94 wt % to 98 wt %.

According to another embodiment, the conversion of methanol is above 98 wt % to less than 100 wt %. According to another embodiment, the conversion of methanol is from 98.1 wt % to less than 100 wt %; preferably from 98.2 wt % to 99.8 wt %. According to another embodiment, the conversion of methanol is from 98.2 wt % to less than 99.5 wt %; preferably from 98.2 wt % to 99wt %.

The 1-olefin produced through catalytic isomerization in accordance with one embodiment of the present invention is polymerized to form a linear low density polyethylene. Methods for polymerizing 1-butene as a co-monomer are well known in the art and are disclosed, for example, in U.S. Pat. Nos. 4,239,871 to Fukui and 5,037,908 to Tachikawa et al., and in Statutory Invention Registration No. H1,254 to Mostert, the entirety of which are incorporated herein by reference. Preferably, a portion of the ethylene produced in an OTO process is passed to a polymerization zone containing polymerization catalyst. In the polymerization zone, 1-butene produced in accordance with the present invention and the ethylene from the OTO process are polymerized at effective conditions to form polyethylene product stream. The polyethylene product comprises a linear low density polyethylene. In another polymerization method well known in the art, 1-butene is a monomer which can be polymerized to form polybutylene.

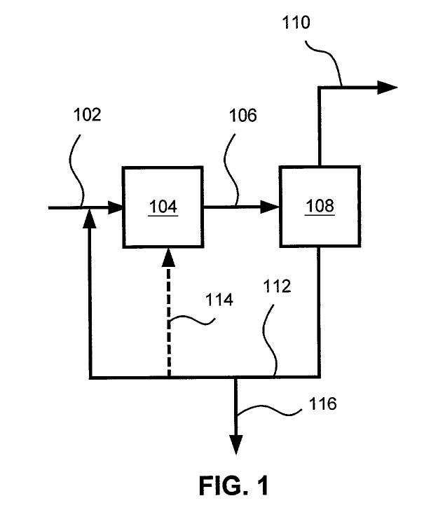

One embodiment of the present invention is shown in FIG. 1. Mixed olefin feedstock stream 102 preferably produced by an OTO unit or a gas cracking unit is shown being directed to an isomerization unit 104. In the isomerization unit 104, the feedstock contacts a small pore molecular sieve catalyst under conditions effective to isomerize at least a portion of the mixed olefin feedstock to 1-olefin. Isomerized stream 106, which includes 1-olefin, is directed to a separation unit 108. The separation unit 108 separates the isomerized stream 106 into a product stream 110 containing mostly 1-olefin and a bottoms stream 112, which can contain one or more of the following: unisomerized cis and trans internal olefin, isoolefin, inerts such as saturated hydrocarbons, dienes, and a small amount of 1-olefin. Preferably, the inerts are periodically or continuously removed from bottoms stream 112 through purge stream 116 to maintain the inerts balance in isomerization unit 104 to a specific compositional range which in turn maintains the olefins at a desired concentration upon introduction to the isomerization unit 104. The purge stream 116 contains parafins such as for example, n-butane or isobutane. The remainder of the bottoms stream is combined with the mixed olefins feedstock stream 102 as shown in bottoms stream 112 of FIG. 1. Alternatively, at least a portion of the bottoms stream is directed to the isomerization unit 104 without being combined with the mixed olefin feedstock stream 102 prior to introduction into isomerization unit 104, as shown by phantom line 114. Optionally, the embodiment disclosed in FIG. 1 includes one or more diene removal units and/or isoolefin removal units, as discussed below.

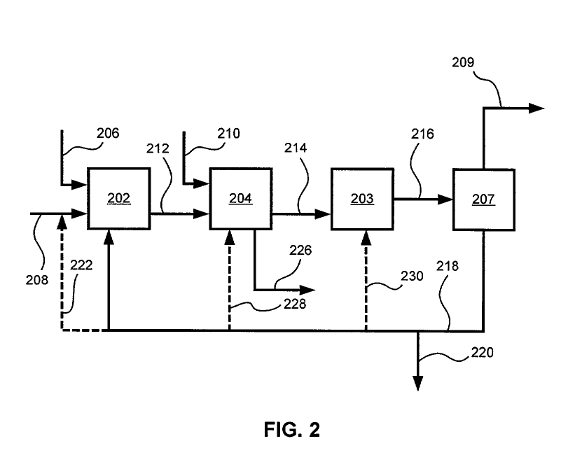

Another embodiment of the present invention is shown in FIG. 2 provides a way of processing a first mixed olefin feedstock stream containing isoolefins, and/or dienes. The first mixed olefin feedstock stream 208 can initially be directed to a diene removal unit 202, wherein at least a portion of the dienes are removed from the first mixed olefin feedstock stream 208. The diene removal unit can be a diene hydrofiner. Hydrogen is added along a hydrogen stream 206 in the presence of a catalyst to convert the dienes to olefins having a like carbon number. As a result, the second mixed olefin feedstock stream 212 contains less diene than the first mixed olefin feedstock stream 208.

As shown in FIG. 2, the second mixed olefin feedstock stream 212 can then be directed to an isoolefin removal unit 204 such as an etherification unit. In an etherification unit, an alcohol stream 210 is directed to the isoolefin removal unit 204 and reacted with the isoolefin in the second mixed olefin feedstock stream 212 conditions effective to form an alkyl ether. The alkyl ether can then be separated through known separation techniques and removed from the mixed olefin feedstock stream as shown in ether removal line 226. Accordingly, third olefin feedstock stream 214 contains less isoolefin than the first and second mixed olefin feedstock streams 208, 212, respectively. Third olefin feedstock stream 214 is directed to the isomerization unit 203.

In the isomerization unit 203, the feedstock contacts preferably a small pore molecular sieve catalyst under conditions effective to isomerize at least a portion of the mixed olefin feedstock to 1-olefin. Isomerized stream 216, which includes 1-olefin, is directed to a separation unit 207 (such as one or more distillation towers) to separate most of the 1-olefin into product stream 209 containing mostly 1-olefin and a bottoms stream 218, which can contain one or more of the following: unisomerized cis and trans internal olefin, isoolefin, inerts such as saturated hydrocarbons, dienes, and a small amount of 1-olefin. If inerts such as parafins are present, they can be removed from bottoms stream 218 through purge stream 220 to maintain the inerts balance in isomerization unit 203 to a specific compositional range. The purge stream 220 can be directed to a second separation system whereby the paraffins, e.g., n-butane and/or isobutane, can be separated from at least a portion of the olefins in the purge stream, for example by extraction, distillation, or other known separation techniques. The separated olefins from the purge line can then be directed to any point in the inventive scheme, e.g., to the diene removal unit, the isoolefin removal unit, the isomerization unit, or any of the lines connecting these units. The separated paraffins can be used to form solvents or gasoline compositions.

The bottoms stream can then be combined with the first mixed olefins feedstock stream 208 as shown in phantom stream 222 of FIG. 2. Additionally or alternatively, at least a portion of the bottoms stream 218 can be directed to the diene removal unit 202 without being combined with the first mixed olefin feedstock stream 208 prior to introduction into diene removal unit 202, as shown by bottoms stream 218. Additionally or alternatively, at least a portion of the bottoms stream 218 can be directed to the isoolefin removal unit 204, as shown by phantom stream 228. Optionally, the at least a portion of the bottoms stream 218 can be directed to the second mixed olefin feedstock stream 212. Additionally or alternatively, at least a portion of the bottoms stream 218 can be directed to the isoolefin removal unit 204, as shown by phantom stream 228. Optionally, the at least a portion of the bottoms stream 218 can be directed to the second mixed olefin feedstock stream 212. In another embodiment, the at least a portion of the bottoms stream 218 can, additionally or alternatively, be directed to isomerization unit 203, as shown in phantom stream 230. Optionally, the at least a potion of the bottoms stream 218 can be directed to the third olefin feedstock stream 214. The proportion of the bottoms stream 218 that is directed to one or more of the purge stream 220, the first, second or third feedstock streams, the diene removal unit 202, the isoolefin removal unit 204 and/or the isomerization unit 203 can be varied in order to arrive at olefin concentrations specifically suited for achieving the best reaction conditions possible in one or more of the diene removal unit 202, isoolefin removal unit 204, and/or isomerization unit 203.

Although FIG. 2 illustrates a feedstock passing from a diene removal unit to an isoolefin removal unit, the feedstock can be passed through the isoolefin removal unit and then through the diene removal unit. Similarly, the feedstock can pass through the isomerization unit before passing through one or both of the diene removal unit and/or the isoolefin removal unit. Thus, the isomerization can be oriented between the diene removal unit and the isoolefin removal unit. In another embodiment, the isomerization unit can be coupled with either, but not necessarily both, the diene removal unit or the isoolefin removal unit.

In one preferred embodiment, for example, a diene removal unit is oriented between the isomerization unit and the separation unit. The removal unit converts at least a portion of the dienes which can be formed by the conditions in the isomerization unit to monoolefin. In this embodiment, an isoolefin removal unit preferably is oriented upstream of the isomerization unit. Because the present invention provides increased selectivity to 1-olefin over isoolefin, the bottoms stream from the separation unit can be recycled, at least in part, to the isomerization unit without being directed to the isoolefin removal unit. In other embodiments, the isoolefin removal unit can be oriented downstream of the isomerization unit.

This invention will be better understood with reference to the following examples, which are intended to illustrate specific embodiments within the overall scope of the invention as claimed.

### EXAMPLES 1, 3 AND 5

A fluid-bed catalyst was formulated from 40 wt % SAPO-34 with a Si/Al₂ ratio of about 0.32 and 60 wt % binder materials. After air calcination at 530° C., the resulting catalyst had a surface area of 350m²/gm, an n-hexane sorption of 40 mg/g, and an alpha value of 1. About 50 mg of this catalyst was diluted in about 2 g of sand and loaded into a fixed-bed, down-flow reactor. The catalyst was pressurized in flowing nitrogen to reaction pressure and pre-heated to the reaction temperature. A mixed olefin feedstock of cis and trans 2-butene was introduced in to the reactor. Reactor product composition was monitored by on-line gas chromatographic analysis on a HP 5890 gas chromatograph having an isoobutene detection limit of about 0.1 weight percent. The operating conditions and isomerized product compositions of the isomerization processes are listed in Table 1.

### EXAMPLES 2 and 4

A fixed-bed catalyst was formulated from 65 wt % ZSM-5 with a silica to alumina ratio of about 25, and 35 wt % alumina binder. After air calcination at 550° C. and steaming 1450° C. for 1 hour, the resulting catalyst had an n-hexane sorption of 60 mg/g, a d/r² of 1900, and an alpha value of 4. About 10 mg was sized to 14/40 mesh, diluted in 2 of sand and loaded into a fixed-bed, down-flow reactor. The catalyst was pressurized in flowing nitrogen to reaction pressure and pre-heated to the reaction temperature. A mixed olefin feedstock of cis and trans 2-butene was introduced in to the reactor. Reactor product composition was monitored by on-line gas chromatographic analysis. The operating conditions and isomerized product compositions of the isomerization processes are listed in Table 1.

### EXAMPLE 6

SAPO-11 was prepared according to the disclosure in U.S. Pat. No. 6,294,493 B1 Strohmaier et al., the entirety of which is incorporated herein by reference. The SAPO-11 catalyst was calcined in air at 525° C. to remove the amine templates thereby activating the catalyst. About 2 mg of the calcined SAPO-11 was mixed with about 2 g of 14/40 mesh sand and loaded into a ⅜ inch stainless fixed-bed reactor. Isco syringe pumps were used to supply cis and trans-2-butene to the reactor. The reactor effluent was analyzed by GC. The operating conditions and isomerized product compositions of the isomerization process is listed in the Table, below.

### EXAMPLE 7

Commercial ZSM-35 pentene skeletal isomerization catalyst was obtained comprising 65 weight percent ZSM-35 and 35 weight percent silica and calcium exchanged to reduce acid activity to about 44 alpha. The ZSM-35 crystal size was about 0.2 micron. Isco syringe pumps were used to supply cis and trans-2-butene to the reactor. A mixed olefin feedstock of cis and trans 2-butene was introduced in to the reactor. Reactor product composition was monitored by on-line gas chromatographic analysis. The operating conditions and isomerized product compositions of the isomerization processes are listed in Table 1.

TABLE

| Example: | 1 | 2 | 3 | 4 | 5 | 6 | 7 |
|---|---|---|---|---|---|---|---|
| Isomerization | | | | | | | |
| Conditions: | | | | | | | |
| Catalyst | SAPO-34 | ZSM-5 | SAPO-34 | ZSM-5 | SAPO-34 | SAPO-11 | ZSM-35 |
| Temp | 480 | 480 | 480 | 480 | 530 | 530 | 480 |
| Pressure, psia | 40 | 40 | 40 | 40 | 15 | 15 | 40 |
| WHSV | 60 | 4800 | 60 | 9600 | 6 | 75 | 1500 |
| Product | | | | | | | |
| Composition: | | | | | | | |
| 1-butene + isobutene | 27.222 | 27.175 | 23.438 | 23.042 | 30.124 | 29.320 | 34.395 |
| Cis-2-butene | 41.797 | 38.738 | 45.733 | 42.939 | 39.177 | 40.122 | 29.192 |
| Trans-2-butene | 29.658 | 28.264 | 30.129 | 29.890 | 29.444 | 29.658 | 42.182 |
| C₅—C₉ non-aromatic | 0.535 | 3.102 | 0.189 | 2.612 | 0.141 | 0.143 | 0.338 |
| Aromatics | 0.055 | 0.187 | 0.018 | 0.304 | 0.016 | 0.000 | 0.006 |
| Product | | | | | | | |
| Selectivities: | | | | | | | |
| 1-butene selectivity | 96.7% | 79.2% | 98.7% | 83.9% | 97.2% | 98.3% | 94.6% |
| Isobutene selectivity | 0.0% | 4.2% | 0.0% | 2.2% | 0.0% | 0.0% | 0.0% |

The data in the Table demonstrates the relatively high product selectivity to 1-butene of the process of the invention, especially when compared to the low selectivities of isobutene. As shown, the SAPO molecular sieve catalysts SAPO-11 and SAPO-34 produce less isobutene than the zeolitic catalysts. Small pore molecular sieve catalyst SAPO-34 is particularly preferred. Although the calcium exchanged ZSM-35 catalyst performed very well (no detectable isobutene), it still produced almost 3 times more byproducts than SAPO-34 and SAPO-11. When ZSM-35 was run with a 10-fold increase in catalyst charge, isobutene yield approached 10 weight percent. When SAPO-34 was run with a 10-fold increase in catalyst charge, isobutene yield remained near the detection limit.

Having now fully described this invention, it will be appreciated by those skilled in the art that the invention can be performed within a wide range of parameters within what is claimed, without departing from the spirit and scope of the invention.

1. A method of isomerizing an olefin feedstock, comprising:
contacting the olefin feedstock with a small pore molecular sieve catalyst under conditions effective to isomerize at least a portion of the olefin feedstock to 1-olefin.

2. The method of claim 1, wherein the 1-olefin is selected from the group consisting of 1-butene, 1-pentene, 1-hexene, 1-heptene, 1-octene, and mixtures thereof.

3. The method of claim 1, wherein the 1-olefin is 1-butene.

4. The method of claim 3, wherein the conditions are effective to provide a 1-olefin:isoolefin conversion index greater than 1:1.

5. The method of claim 4, wherein the 1-olefin:isoolefin conversion index is greater than 5:1.

6. The method of claim 5, wherein the 1-olefin:isoolefin conversion index is greater than 10:1.

7. The method of claim 6, wherein the 1-olefin:isoolefin conversion index is greater than 20:1.

8. The method of claim 7, wherein the 1-olefin:isoolefin conversion index is greater than 50:1.

9. The method of claim 3, wherein the conditions include a temperature of at least 350° C.

10. The method of claim 3, wherein the conditions include a temperature of from 300° C. to 700° C.

11. The method of claim 10, wherein the temperature is from 400° C. to 650° C.

12. The method of claim 11, wherein the temperature is from 450° C. to 600° C.

13. The method of claim 12, wherein the temperature is from 450° C. to 550° C.

14. The method of claim 3, wherein the conditions include a butenes partial pressure of from 5 psia to 150 psia.

15. The method of claim 3, wherein the conditions include a WHSV of from 1 hr⁻¹ to 1000 hr⁻¹.

16. The method of claim 3, wherein less than 2% by weight of the olefin feedstock is converted to hydrocarbons with a higher carbon number.

17. The method of claim 3, wherein less than 1% by weight of the olefin feedstock is converted to hydrocarbons with a higher carbon number.

18. The method of claim 1, wherein the small pore molecular sieve catalyst is a small pore silicoaluminophosphate molecular sieve catalyst (SAPO).

19. The method of claim 18, wherein the SAPO is selected from the group consisting of: SAPO-17, SAPO-18, SAPO-34, SAPO-35, SAPO-44, the substituted forms thereof, and mixtures thereof.

20. The method of claim 19, wherein the SAPO is SAPO-34.

21. The method of claim 1, wherein the olefin feedstock comprises olefin produced by a gas cracking unit, an oxygenate to olefin unit, or a mixture thereof.

22. The method of claim 1, wherein the olefin feedstock is an isoolefin depleted feedstock.

23. The method of claim 1, wherein the olefin feedstock comprises less than 2% by weight isoolefin.

24. The method of claim 23, wherein the olefin feedstock comprises less than 1% by weight isoolefin.

25. The method of claim 1, wherein less than 2% by weight of the olefin feedstock is converted to aromatic hydrocarbons.

26. The method of claim 3, wherein the olefin feedstock includes butadiene, the method further comprising:
contacting the butadiene with hydrogen under conditions effective to convert at least a portion of the butadiene to C₅⁺ compounds.

27. The method of claim 26, further comprising:
separating the C₅⁺ compounds from the olefin feedstock.

28. The method of claim 27, wherein the olefin feedstock includes isoolefin, the method further comprising:
contacting the isoolefin with an alcohol under conditions effective to convert at least a portion of the isoolefin to an alkyl ether.

29. The method of claim 28, further comprising:
separating the alkyl ether from the olefin feedstock.

30. The method of claim 3, wherein the olefin feedstock includes isoolefin, the method further comprising:
contacting the isoolefin with an alcohol under conditions effective to convert at least a portion of the isoolefin to an alkyl ether.

31. The method of claim 30, further comprising:
separating the alkyl ether from the olefin feedstock.

32. The method of claim 3, wherein the olefin feedstock includes 2-butene.

33. The method of claim 32, wherein the olefin feedstock includes 1-butene.

34. The method of claim 33, wherein the olefin feedstock includes isobutene.

35. A method of isomerizing an olefin feedstock, comprising:
contacting the olefin feedstock with a molecular sieve catalyst under conditions effective to isomerize at least a portion of the olefin feedstock to 1-olefin, wherein the conditions include a temperature of at least 300° C. and the contacting provides a 1-olefin:isoolefin conversion index of greater than 1:1.

36. The method of claim 35, wherein the temperature is from 300° C. to 700° C.

37. The method of claim 36, wherein the temperature is from 400° C. to 650° C.

38. The method of claim 37, wherein the temperature is from 450° C. to 600° C.

39. The method of claim 38, wherein the temperature is from 450° C. to 550° C.

40. The method of claim 35, wherein the 1-olefin:isoolefin conversion index is greater than 5:1.

41. The method of claim 40, wherein the 1-olefin:isoolefin conversion index is greater than 10:1.

42. The method of claim 41, wherein the 1-olefin:isoolefin conversion index is greater than 20:1.

43. The method of claim 42, wherein the 1-olefin:isoolefin conversion index is greater than 50:1.

44. The method of claim 43, wherein the catalyst is selected from the group consisting of: SAPO-34, SAPO-11, ZSM-35, ZSM-5 and Ferrierite.

45. The method of claim 44, wherein the catalyst includes SAPO-34.

46. The method of claim 44, wherein the catalyst includes SAPO-11.

47. The method of claim 46, wherein the SAPO-11 comprises ECR-42.

48. The method of claim 35, wherein the olefin feedstock is formed by a gas cracking unit, an oxygenate to olefin unit or a mixture thereof.

49. The method of claim 35, wherein the olefin feedstock is a C₄⁺ fraction produced by an oxygenate to olefin unit.
